SEQUENCE LISTING (4 sequences)

SEQ ID NO: 1 - DNA, 74 bp, synthetic construct
  source 1..74: mol_type = other DNA
aatgatacggcgaccaccgagatctacacatggccgactacactctttccctacacgacgctcttccgatcttt

SEQ ID NO: 2 - DNA, 18 bp, synthetic construct
  source 1..18: mol_type = other DNA
gatcggaagagcgtcgtg

SEQ ID NO: 3 - DNA, 58 bp, synthetic construct
  source 1..58: mol_type = other DNA
agatcggaagagcacacgtctgaactccagtcacatctcgtatgccgtcttctgcttg

SEQ ID NO: 4 - DNA, 19 bp, synthetic construct
  source 1..19: mol_type = other DNA
cgtgtgctcttccgatctt

(19) United States
(12) Patent Application Publication
(10) Pub. No.: US 2026/0193695 A1
(43) Pub. Date: Jul. 9, 2026

(54) METHOD OF AMPLIFYING NUCLEIC ACID BY POLYMERASES WITH STRAND DISPLACEMENT ACTIVITY

(71) Applicant: QIAGEN GmbH, Hilden (DE)

(72) Inventor: Ioanna ANDREOU, Koeln (DE)

(21) Appl. No.: 19/134,301

(22) PCT Filed: Nov. 17, 2023
(86) PCT No.: PCT/EP2023/082281

§ 371 Date: May 30, 2025

(30) Foreign Application Priority Data
Dec. 6, 2022 (EP) 22211701.2

(51) Int. Cl. C12Q 1/6844; C12Q 1/6806; C12Q 1/6869

(52) U.S. Cl. CPC C12Q 1/6844; C12Q 1/6806; C12Q 1/6869

(57) ABSTRACT

The present invention relates to a new agent to modulate nucleic acid amplification by a polymerase with stand displacement activity, a method of amplifying nucleic acid by a polymerase with stand displacement activity involving said agent, a method of preparing a nucleic acid sequencing library, a method of sequencing a nucleic acid, and a kit for amplifying nucleic acid amplification by a polymerase with stand displacement activity.

The present invention relates to a new agent to modulate nucleic acid amplification by a polymerase with strand displacement activity, a method of amplifying nucleic acid by a polymerase with strand displacement activity involving said agent, a method of preparing a nucleic acid sequencing library, a method of sequencing a nucleic acid, and a kit for amplifying nucleic acid amplification by a polymerase with strand displacement activity.

### FIELD OF THE INVENTION

The present invention relates to the field of molecular biology, more particularly to the amplification of nucleic acid molecules, and, specifically, to the amplification of nucleic acid by a polymerase with strand displacement activity.

### BACKGROUND OF THE INVENTION

Strand displacement amplification (SDA) is a method for the targeted amplification of nucleic acids and an alternative to the classical polymerase chain reaction. It is based on the ability of some DNA polymerases to start new synthesis at a single-strand break and thereby displace the old strand, so-called strand displacement.

The most prominent SDA is the so-called multiple displacement amplification (MDA). This method can rapidly amplify minute amounts of DNA samples to a reasonable quantity for genomic analysis. The reaction starts by annealing random hexamer primers to the template: DNA synthesis is carried out by a high-fidelity enzyme, preferentially 029 DNA polymerase.

Compared with conventional PCR amplification techniques, typically SDA or MDA does not employ sequence-specific primers but amplifies all DNA. Instead, it uses hexamer primers, i.e., primer sequences composed of six random nucleotides. The amplification reaction initiates when multiple primer hexamers anneal to the template. When DNA synthesis proceeds to the next starting site, the polymerase displaces the newly produced DNA strand and continues its strand elongation. The strand displacement generates a newly synthesized single-stranded DNA template for more primers to anneal. Further primer annealing and strand displacement on the newly synthesized template results in a hyper-branched DNA network. To separate the DNA branching network, S1 nucleases are used to cleave the fragments at displacement sites. The nicks on the resulting DNA fragments are repaired by DNA polymerase I.

SDA and MDA generate larger-sized products with a lower error frequency, and works at a constant temperature. SDA and MDA have been actively used in whole genome amplification (WGA) and is a promising method for application to single cell genome sequencing and sequencing-based genetic studies; see Lovmar and Syvänen (2006), Multiple displacement amplification to create a long-lasting source of DNA for genetic studies, Hum. Mutat. 27(7), pp. 603-614. It generates hyperbranched long DNA fragments with high molecular weight of approx. 20-50 Kb.

This long DNA fragments that results from SDA need fragmentation prior library generation, something that is time consuming and lead to DNA loss and increase sequencing bias. Currently such methods are optimized for sequencing platforms which allow short fragment sequencing with fragment length from 50 bp to 550 bp.

However due to high molecular weight of DNA produced by the SDA reaction long fragment sequencing is feasible. One of the main drawback of this material is its hyperbranchity of amplified DNA, that will clog the pores on sequencing apparatuses during sequencing, such as Nanopore® sequencers. There are additional protocols and that describe purifications steps and/or enzymatic fragmentation of whole genome amplification (WGA) DNA prior to library generation.

According to a workflow proposed by Nanopore® for sequencing VGA DNA generated using, for example, QIAGEN REPLI-g Midi kit is treated after amplification with T7 endonuclease I, which resolves the hyperbranched structure of the VGA product and allows to obtain average quality scores which are similar to those obtained without WGA. The treatment with T7 endonuclease I is followed by cleanup steps and size selection using magnetic beads.

In the art, it is described that this digestion procedure and cleanup depending on its efficiency will lead either to DNA loss or to insufficient linearization of the hyperbranched DNA and blocking of the pores of the sequencers. Depending on the input quality of the WGA reaction and the resulted yields, molecular weight and hyperbranchity of amplified DNA, optimization of the workflow is needed. The result of optimization is only visual after sequencing and is some case will lead to complete loss of sample, without obtaining any valuable sequencing results.

Another method to prepare WGA-DNA prior to Nanopore sequencing is mechanical shearing as described in Ye et al. (2020), A high-quality de novo genome assembly from a single parasitoid wasp, bioRxiv 2020.07.13.200725. This technique requires additional equipment and also higher input of DNA in every step of the workflow.

Against this background, it is the task of the present invention to improve the currently performed SDA reactions in such a way that the disadvantages outlined above are avoided or at least reduced. In particular, the amplification methods currently used, in which polymerases with strand displacement activity are employed, are to be modified in such a way that the currently required time-consuming and costly steps up to the production of a sequencing library can be reduced or even eliminated.

The present invention satisfies these and other needs.

### SUMMARY OF THE INVENTION

The present invention provides the use of an inorganic lithium and/or magnesium salt to modulate nucleic acid amplification by a polymerase with strand displacement activity.

This object is also met by a method of amplifying nucleic acid comprising (i) the provision of nucleic acid in a reaction environment, and (ii) amplifying said nucleic acid, wherein said reaction environment comprises a polymerase with strand displacement activity and inorganic lithium and/or magnesium salt.

In principle, all inorganic lithium and/or magnesium salts and mixtures thereof are suitable according to the invention. According to the findings of the inventor, such inorganic lithium salts and/or inorganic magnesium salts are particularly preferred, each having anions selected from the group consisting of chloride, oxide, bromide, iodide, carbonate, sulfide, sulfate, phosphate, and nitrate, further preferably chloride, bromide, iodide, and most preferably chloride, i.e., LiCl and/or MgCl.

Against this background, not only inorganic lithium salts or magnesium salts can be used, but also any mixtures thereof.

“Strand displacement activity” is the ability of polymerases, in particular DNA polymerase, to start with new synthesis at a single-strand break of a nucleic acid, thereby displacing the opposite strand.

Consequently, according to the invention, the phrase “nucleic acid amplification by a polymerase with strand displacement activity” can be replaced by “strand displace amplification” (SDA). Both phrases have the same meaning according to the invention and can be used interchangeably.

Encompassed according to the invention are all polymerases having strand displacement activity, including, but not limited to, those used in the context of whole genome amplification (WGA). Particularly encompassed, therefore, are φ29 DNA polymerase, Bst DNA polymerase, Bst DNA polymerase large fragment, and Taq DNA polymerase mutant (SD DNA polymerase).

“Modulation” or “modulate” according to the invention comprises any interference with the reaction and/or activity of the strand displacement polymerase, in particular an interference that leads to the reduction of the lengths and/or the degree of (hyper-) branching of the amplified nucleic acid molecules.

“Nucleic acid” according to the invention includes any kind of nucleic acid, especially desoxyribonucleic acid (DNA) and ribonucleic acid (RNA), in particular double-stranded nucleic acid (dsDNA, dsRNA). The nucleic acid can be of any origin, such as prokaryotic and eukaryotic, of animal, plant, bacterial or viral origin etc., from mammals, humans or other living organisms.

According to the invention, a “reaction environment” is understood to be a medium in which the polymerase reaction takes place. A reaction environment can therefore be a reaction mixture, e.g., a reaction buffer, or a reaction solution or liquid to which the inorganic lithium and/or magnesium salt is added.

In particular, according to the invention, ‘multiple displacement amplification’ (MDA) is envisaged, which can be significantly improved by the addition of inorganic lithium and/or magnesium salt. MDA is a nucleic acid amplification technique which can rapidly amplify minute amounts of preferentially DNA samples to a reasonable quantity for genomic analysis. The reaction starts by annealing random hexamer primers or, alternatively, target-specific primers or a mixture of random and target-specific primers to the template: DNA synthesis is carried out by a high-fidelity polymerase with strand displacement activity, preferentially φ29 DNA polymerase. Compared with conventional PCR amplification techniques, MDA does not generate amplicon between two sequence-specific primers but amplifies all DNA, generates larger-sized products with a lower error frequency, and works at a constant temperature. MDA has been actively used in whole genome amplification (WGA) and is a promising method for application to single cell genome sequencing and sequencing-based genetic studies.

The inventor has realized that the addition of an inorganic lithium and/or magnesium salt to a nucleic acid amplification reaction carried out by a polymerase with strand displacement activity results in smaller and less (hyper-) branched nucleic acid fragments. Said fragments can be directly used for generating a sequencing library, including end-repairing and/or adapter ligation. The method and use according to the invention do not require steps of fragmenting the amplified nucleic acid, cleaning and washing steps required to remove enzyme(s) and reactants used for the fragmenting procedure. Thus, a number of processing steps can be omitted. This significantly reduces the risk of contamination or loss of amplified nucleic acid.

The invention, therefore, allows one-step library generation from single cells and low input, and sequencing of long reads, for example on Nanopore® instruments.

Finally, the inventor has found out, that when adding inorganic lithium and/or magnesium salts to the amplification reaction mediated by polymerases with strand displacement activity the length or length distribution of the amplified nucleic acid fragments and/or the degree of (hyper-) branching can be controlled and modulated.

The problem underlying the invention is hereby completely solved.

According to an embodiment of the use and method of the invention said modulation results in the generation of nucleic acid amplicons that are smaller compared to nucleic acid amplicons generated in a reference nucleic acid amplification using a polymerase with strand displacement activity without inorganic lithium and/or magnesium salt.

According to another embodiment of the use and method of the invention said modulation results in the generation of nucleic acid amplicons that are less branched compared to nucleic acid amplicons generated in a reference nucleic acid amplification using a polymerase with strand displacement activity without inorganic lithium and/or magnesium salt.

According to the invention, a “reference nucleic acid amplification” refers to such a reaction environment that is identical to the reaction environment according to the invention, but does not include inorganic lithium and/or magnesium salt.

The inventor has found that the addition of inorganic lithium and/or magnesium salt alone allows control over the fragment lengths or fragment length distribution as well as the degree of (hyper-) branching of the amplified nucleic acid molecules, i.e., leads to shorter fragment lengths and less branching.

In a still further embodiment of the use and method of the invention said inorganic lithium salt is selected from the group consisting of: lithium chloride (LiCl), lithium oxide (Li₂O), lithium bromide (LiBr), lithium iodide (LiI), lithium carbonate (Li₂CO₃), lithium sulfite (Li₂SO₃), lithium sulfate (Li₂SO₄), lithium phosphate (Li₃PO₄), and lithium nitrate (LiNO₃).

In yet another embodiment of the use and method of the invention said inorganic magnesium chloride is selected from the group consisting of: magnesium chloride (MgCl), magnesium oxide (MgO), magnesium bromide (MgBr₂), magnesium iodide (MgI₂), magnesium carbonate (MgCO₃), magnesium sulfite (MgSO₃), magnesium sulfate (MgSO₄), magnesium phosphate (Mg₃(PO₄)₂), and magnesium nitrate (Mg(NO₃)₂).

This measure provides such salts that the inventor has found to be quite suitable. For example, the inventor has been able to experimentally validate the suitability of organic salts for modulating the activity of polymerases with strand displacement activity mainly using a model lithium salt, in particular LiCl. However, due to, among other things, the so-called oblique relationship in the periodic table, the skilled person can understand that lithium and magnesium salts are equally suitable in the context of the present invention.

In still another embodiment of the use and method of the invention said nucleic acid is double-stranded desoxyribonucleic acid (dsDNA) and/or double-stranded ribonucleic acid (dsRNA).

This measure has the advantage that the inorganic lithium and/or magnesium salt according to the invention can be used in the amplification of such nucleic acid molecules for which strand displacement polymerases are particularly suitable. Indeed, dsDNA and dsRNA often form the genomes of organisms or viruses, and strand displacement polymerases have been particularly useful for whole genome amplification (WGA).

Against this background, in another embodiment of the use and method of the invention said polymerase with strand displacement activity is selected from the group consisting of φ29 DNA polymerase, Bst DNA polymerase, Bst DNA polymerase large fragment, and Taq DNA polymerase mutant (SD DNA polymerase).

With this measure, the invention is advantageously adapted to the use of such strand displacement polymerases that have been particularly successful and relevant in practice.

In another embodiment of the use and method of the invention said nucleic acid amplification is whole genome amplification (WGA).

This measure adapts the invention to the optimization of the amplification most significant in practice, which involves polymerases with strand displacement activity.

In a preferred embodiment of the use and method of the invention said inorganic lithium and/or magnesium salt is used in a concentration range from about 1 mM to 100 mM, preferably from about 10 mM to 75 mM, more preferably from about 12.5 mM to 50 mM, and most preferably at a concentration of about 30 mM.

As the inventor was able to determine, the inorganic lithium and/or magnesium salt shows the best effect on the fragment length and the degree of branching of the amplicons in the indicated concentration ranges.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Another subject-matter of the invention is a method of preparing a nucleic acid sequencing library comprising (i) the provision of amplified nucleic acid, and (ii) subjecting said amplified nucleic acid to a nucleic acid sequencing library, characterized in that said amplified nucleic acid is prepared by the method of amplifying nucleic acid according to the invention.

A still further subject-matter of the invention relates to a method of sequencing a nucleic acid (i) the provision of amplified nucleic acid, and (ii) subjecting said amplified nucleic acid to a sequencing reaction, characterized in that said amplified nucleic acid is prepared by the method of amplifying nucleic acid according to the invention.

The features, characteristics, and advantages explicitly disclosed for the use and the method of amplifying nucleic acid according to the invention apply, mutatis mutandis, to the method of preparing a nucleic acid sequencing library and to the method of sequencing a nucleic acid.

Another subject-matter of the invention is a kit for amplifying nucleic acid comprising
a) a first container comprising (i) a polymerase with strand displacement activity, (ii) an inorganic lithium and/or magnesium salt, and (iii) random and/or target-specific primers, each in solution or in lyophilized and/or crystalline formulation;
b) optionally, a second container containing a diluent or reconstituting solution for the lyophilized fand/or crystalline formulation;
c) optionally, instructions for carrying out the methods according to the invention.

The components recited under (i), (ii), and (iii), may, in an embodiment of the invention, also be comprised by separate containers, i.e., a first container contains the polymerase, a second container contains the salt, and a third container contains the primers. In such embodiment the diluent is contained by a fourth container.

The kit may further comprise nucleic acid, one or more of a buffer, a diluent, a filter, a needle, or a syringe. The container(s) is/are preferably a bottle, a vial, a syringe or test tube; and it may be a multi-use container. The polymerase with strand displacement activity is preferably lyophilized or, alternatively, in solution. The container(s) may be formed from a variety of materials such as glass or plastic. Preferably the kit and/or container contain/s instructions on or associated with the container(s) that indicate directions for reconstitution and/or use. For example, the label may indicate that the lyophilized and/or crystalline formulation is to be reconstituted to salt concentrations as described above.

The features, characteristics, and advantages explicitly disclosed for the use and the method of amplifying nucleic acid according to the invention apply, mutatis mutandis, to the kit.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the detailed methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided can be different from the actual publication dates, which can need to be independently confirmed.

### EMBODIMENTS

Before the present invention is further described, it is to be understood that this the disclosure is not strictly limited to particular embodiments described herein, as such can of course vary. It is also to be understood that the terminology used herein is for the purpose of describing particular embodiments only, and is not intended to be limiting, since the scope of the present invention will be limited only by the claims.

The invention is now further explained by means of embodiments resulting in additional features, characteristics and advantages of the invention. The features mentioned in the specific embodiments are features of the invention and may be seen as general features which are not applicable in the specific embodiment but also in an isolated manner in the context of any embodiment of the invention.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

The invention is now further described and explained in further detail by referring to the following non-limiting examples and figures:

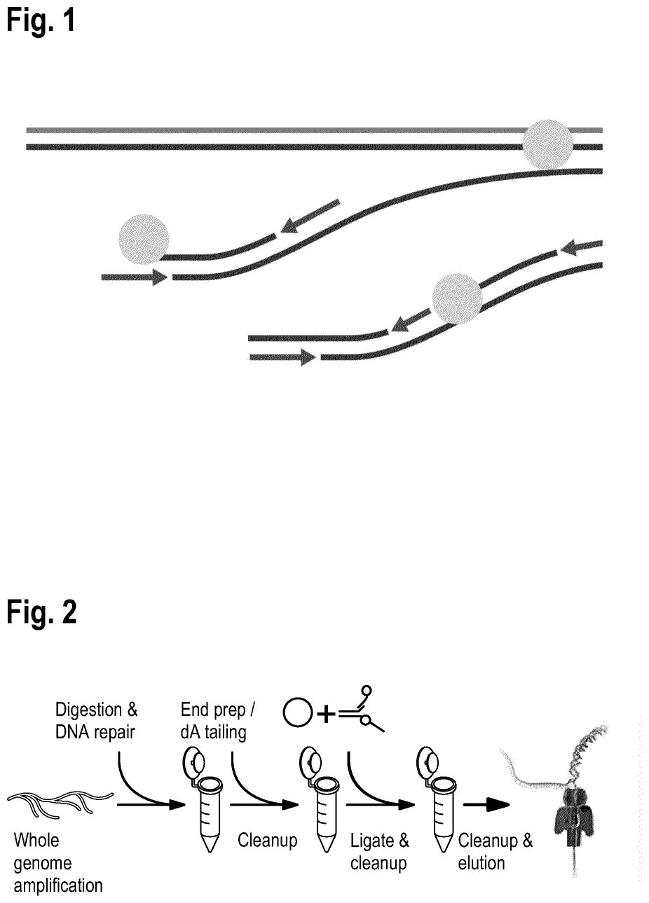

FIG. 1: Illustration of multiple displacement amplification (MDA).

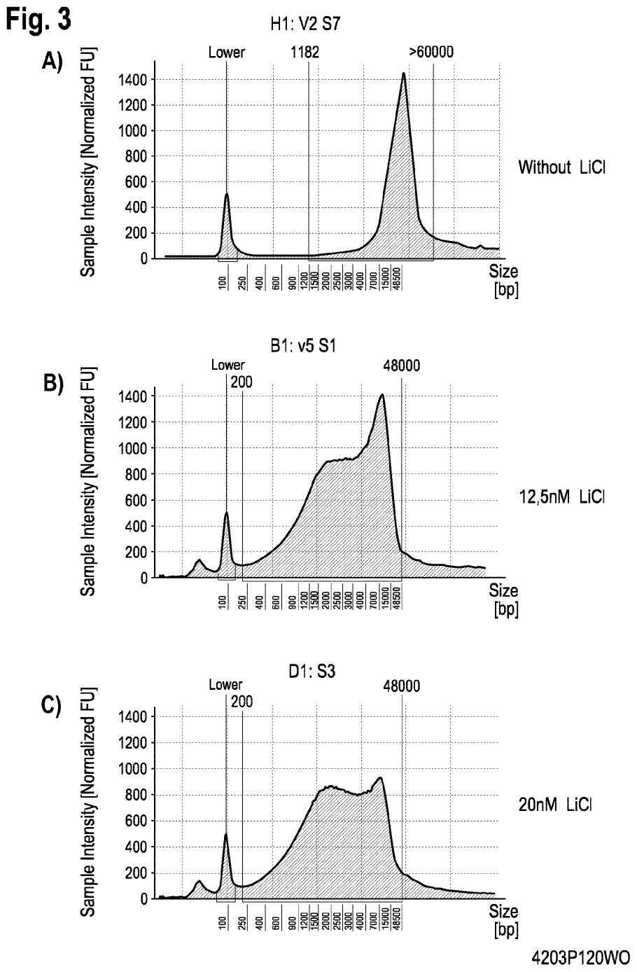

FIG. 2: Workflow of preparation of whole genome amplification (WGA) for Nanopore® sequencing.

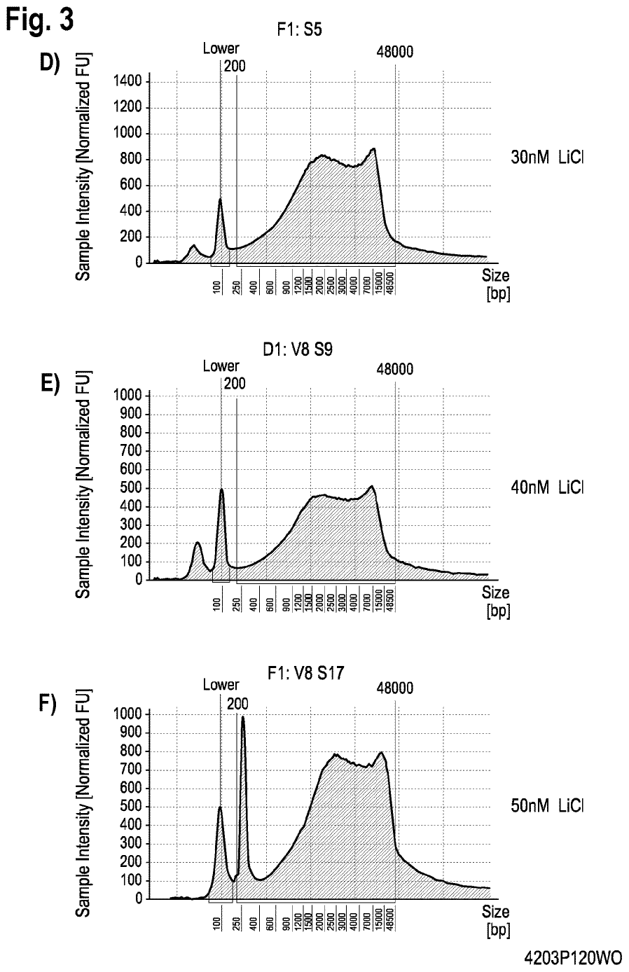

FIG. 3: Fragment size distribution of WGA-DNA in correlation to added LiCl concentration.

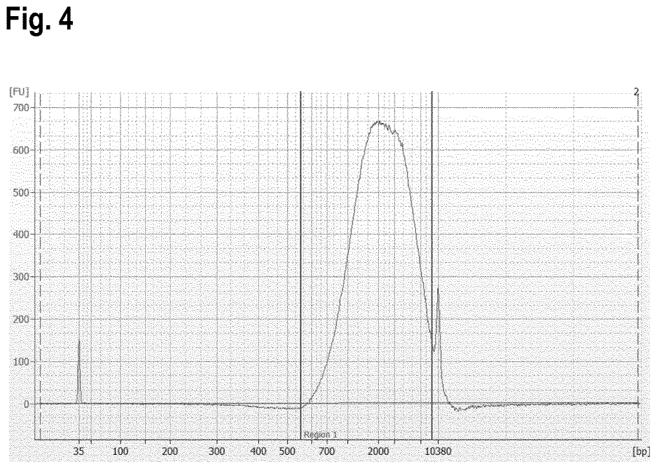

FIG. 4: Library size distribution of a direct library after WGA.

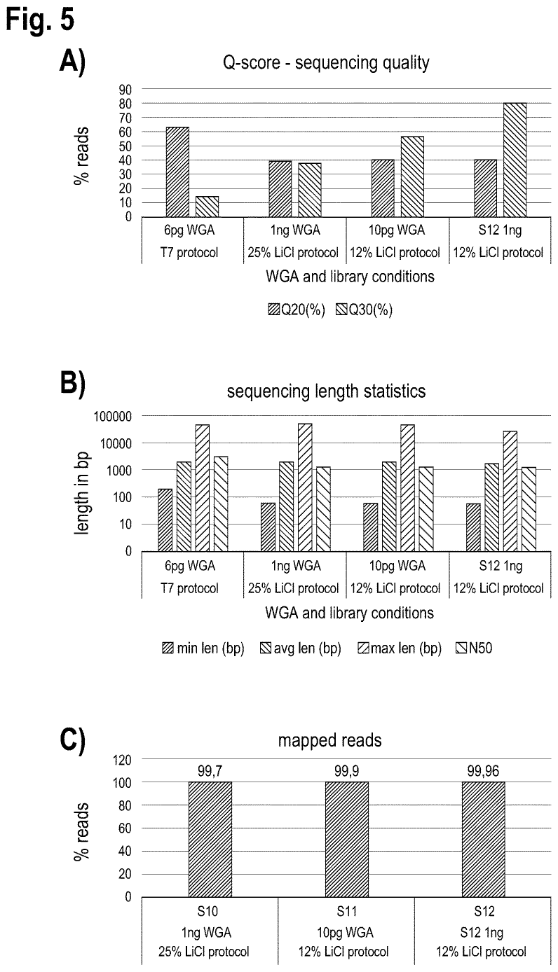

FIG. 5: Comparison with known libraries using T7 treatment.

### EXAMPLE

Multiple Displacement Amplification (MDA) using 029 DNA polymerase efficiently amplifies DNA isothermally, by repeated annealing of random short oligo nucleotides. It generates hyperbranched long DNA fragments with high molecular weight of approx. 20-50 Kb (FIG. 1). In the MDA methods known in the art the long DNA fragments need fragmentation prior library generation and sequencing. The workflow used in whole genome amplification (WGA) for Nanopore® sequencing is depicted in FIG. 2.

According to this workflow, WGA DNA generated using for example QIAGEN REPLI-g Midi kit is treated after amplification with T7 endonuclease I, which resolves the hyperbranched structure of the WGA product and allows us to obtain average Q-scores which are similar to those obtained without WGA. The treatment with T7 endonuclease I is followed by cleanup steps and size selection using magnetic beads.

The invention uses a simple additive during the MDA reaction, which can modulate the activity of reaction and result in smaller and less hyperbranched DNA fragments, which can be directly used for adapter ligation and library generation.

This shortens the protocol, since it eliminates the step of the fragmentation of WGA-DNA and reduce interaction steps which may contaminate the sample and lead to DNA loss.

The inventor has observed that when adding inorganic lithium and/or magnesium salt, e.g., LiCl, in the MDA reaction one can control the fragment length distribution of the amplified DNA (FIG. 3).

This DNA can be directly end-repaired and ligated to adapters to generate a sequencing library without the need of extra fragmentation. The generated libraries have fragment size 700 to >10000 bp and can be used for long read sequencing for Example on Nanopore® platforms.

FIG. 4 demonstrates the size distribution of a library, that was generated directly after WGA using inorganic lithium and/or magnesium salt such as LiCl to reduce fragment size.

### Workflow

WGAs and libraries were performed using the workflow and component described in the following table. The modulation of WGA-DNA molecular weight and hyperbranchity with LiCl is applicable for both WGA workflows, barcoded WGA and standard WGA. The description of both options is included in the following table.

Direct WGA & Library Workflow

| STEP | | | |
|---|---|---|---|
| 1 | Lysis of Cell or denaturation of DNA generation of single | | |
| | cell to allow annealing of WGA barcode over the random portion | | |
| | | DNA or Cell | 1 μl |
| | | Barcode (option 1) or | 1 |
| | | random primer (option 2) | |
| | | add denaturation agent | 1.5 μl |
| | incubate at 65° for 10 min (cell) or 3 min at room temperature | | |
| | (gDNA) | | |
| | WGA-barcode | TdUN₁N₂N₃NNNNN_{3C}N_{2C}N_{1C}AANNNN*N*N | |
| | format | | |
| | random primer | NNNN*N*N | |
| 2 | Neutratlization | | |
| | | add neutralization mix | 1.5 μl |
| | | mix and place on Ice | |
| 3 | RepliG amplification starting from random fraction of WGA-Barcode | | |
| | or random primer | | |
| | | add WGA reaction mix | |
| | | WGA reaction buffer | 14.5 μl |
| | | WGA reaction oligo | variable |
| | | LiCl | Variable; 12,6 mM to 50 mM |
| | | Water | 2.03 μl |
| | | WGA enzyme | 1 μl |
| 4 | Pooling of WGA and purification | | |
| | | pool 10 μl of each 24 MDA | 240 μl |
| | | purify with 1,2x QIAseq Beads | 288 μl |
| | | bind | 5 min at room temperature |
| | | wash | 2x with 200 μl 80% Ethanol |
| | | elute with | 50 μl Water |
| 5 | End polishing Sticky End generation am the 5′ end of WGA-Barcode | | |
| | | Sample from previous step | 20 |
| | | Water (no longer need to use UV | 1.5 |
| | | treated) | |
| | | Endpolishing buffer 10x | 2.5 |
| | | Endpolishing enzyme mix | 1 |
| | | Mix well by pipetting then incubate in thermocycler | |
| | | with heated lid ON; 70-100° C. | |
| | | 30 min | 25° C. |
| | | 15 min | 65° C. |
| | | for ever | 4° C. |
| | | Sample from previous step | 25 μl |
| | | Water | 2.4 μl |
| | | Reaction buffer | 0.6 μl |
| | | UDG | 1.5 μl |
| | | ENDOVIII | 1.5 μl |
| | | Mix well by pipetting then incubate in thermocycler | |
| | | with heated lid ON; 70-100° C. | |
| | | 1 min | 4° C. |
| | | 60 min | 37° C. |
| | | for ever | 4° C. |
| 6_ | I5 and I7 adapter ligation when WGA-Barcodes are used | | |
| option 1 | | add to the 31 μl of reaction from previous step | |
| | | Water | 28 μl |
| | | Ligation buffer | 18 μl |
| | | Adapter 1 | 4.5 μl |
| | | Adapter 2 | 4.5 μl |
| | | T4 DNA Ligase | 4 μl |
| | | Mix very well by pipetting then incubate in | |
| | | thermocycler with heated lid OFF | |
| | | 1 min | 4º C. |
| | | 30 min | 25° C. |
| | | for ever | 4º C. |
| | adapter 1 format | AATGATACGGCGACCACCGAGATCTACACATGGCCGACTAC | |
| | (Illumina compat.) | ACTCTTTCCCTACACGACGCTCTTCCGATCT*T*T (SEQ ID NO: 1) | |
| | | hybridized with GATCGGAAGAGCGTCGTG/3phos/ (SEQ ID NO: 2)) | |
| | adapter 2 format | p-AGATCGGAAGAGCACACGTCTGAACTCCAGTCACin- | |
| | (Illumina compat.) | dex7ATCTCG TATGCCGTCTTCTGCTT*G (SEQ ID NO: 3) | |
| | Illumina compat. | hybridised with CGTGTGCTCTTCCGATCT*T/3Phos/ (SEQ ID NO: 4) | |
| | | *: Phosphothioate | |
| | | NOTE: adapters are partially double strands | |
| 6_ | I5 and I7 adapter ligation, when only random hexamers are used | | |
| option 2 | | add to the 31 μl of reaction from previous step | |
| | | Water | 28 μl |
| | | Ligation buffer | 18 μl |
| | | QIAseq Y-shaped Adapters | 9 μl |
| | | (Illumina compat.) | |
| | | T4 DNA Ligase | 4 μl |
| | | Mix very well by pipetting then incubate in | |
| | | thermocycler with heated lid OFF | |
| | | 1 min | 4º C. |
| | | 30 min | 25° C. |
| | | for ever | 4º C. |
| 7 | Bead purification to remove non ligated adapters | | |
| | | purify with 0,8x QIAseq Beads | 80 μl |
| | | bind | 5 min at room temperature |
| | | wash | 2x with 200 μl 80% Ethanol |
| | | elute with | 50 μl Water |
| | | purify with 0,8x QIAseq Beads | 40 μl |
| | | bind | 5 min at room temperature |
| | | wash | 2x with 200 μl 80% Ethanol |
| | | elute with | 20 μl Water |
| 8 | Library amplification and completion of partially single | | |
| | stranded adapters to double strands | | |
| | | Pipette the HiFi amplification Mix | |
| | | Sample from Ligation | 11.75 |
| | | PCR master mix | 12.5 |
| | | 10 uM platform-specific library | 0.75 |
| | | amplification primer | |
| | | Mix well by pipetting and incubate in | |
| | | thermocycler with heated lid on | |
| | | 2 min | 98° C. |
| | 15-20 times | 20 sec | 98° C. |
| | | 30 sec | 62° C. |
| | | 30 sec | 72° C. |
| | | 1 min | 72° C. |
| | | for ever | 4° C. |
| 9 | Bead purification-final cleanup of the library | | |
| | | add 75 μl ice cold water | |
| | | to make 100 μl | |
| | | purify with 1x QIAseq Beads | 100 μl |
| | | bind | 5 min at room temperature |
| | | wash | 2x with 200 μl 80% Ethanol |
| | | elute with | 20 μl Water |

WGAs were performed using different concentrations of LiCl (UPX Booster). The WGA were ligated to adapter suitable for nanopore sequencing using the ‘Nanopore Protocol’ named ‘Native Barcoding Genomic Dna NBE-9065-v109-revAC-14aug.2019 Minion’.

Generated data were analyzed using CLC analysis tools and were compared to published data from Libraries which were generated from WGA DNA using T7 endonuclease treatment as described in Ni et al. (2023), Benchmarking of Nanopore R10.4 and R9.4.1 flow cells in single-cell whole-genome amplification and whole-genome shotgun sequencing, Computational and Structural Biotechnology Journal 21, 2352-2364.

As can be inferred from the following table and FIG. 5 where the sequencing quality (Q score) (A), the sequencing length statistics (B), and the mapped reads (C) are visualized, the data show performance similar to the library generated using T7 treatment. The fragment length is comparable. % of reads with >Q30 is higher with the libraries generated from WGA with addition of LiCl. The mapping rate is high which supports the unbiased high quality reads.

Filtered data

| sample/ | | min | avg | max | | | |
|---|---|---|---|---|---|---|---|
| amp/ | | length | length | length | | Q20 | Q30 |
| treatment | | (bp) | (bp) | (bp) | N50 | (%) | (%) |
| T7 proto- | 6 pg | 200 | 2004 | 43464 | 2977 | 64 | 15 |
| col | WGA | | | | | | |
| 25% LiCl | 1 ng | 57 | 1918 | 49727 | 1290 | 39 | 38 |
| protocol | WGA | | | | | | |
| 12% LiCl | 10 pg | 58 | 1990 | 47090 | 1289 | 40 | 57 |
| protocol | WGA | | | | | | |
| 12% LiCl | S12 | 60 | 1673 | 26763 | 1312 | 40 | 80 |
| protocol | 1 ng | | | | | | |

In case of discrepancies between the sequences given in the present description and those given in the sequence listing, the sequences from the description shall prevail.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### CONCLUSION

The inventor provides a method that is easy to establish in practice and simple to use, with which the numerous procedural steps and the associated risk of nucleic acid loss previously required in ‘whole genome sequencing’, or, more generally, in methods in which polymerases with strand displacement activity are used, can be reduced. The invention can be easily integrated into existing procedures without the need for elaborate adaptations of established protocols.

1-22. (canceled)

23: A method for modulating nucleic acid amplification by a polymerase with strand displacement activity comprising providing an inorganic lithium salt and/or an inorganic magnesium salt in a reaction environment that comprises a nucleic acid and the polymerase with strand displacement activity.

24: The method according to claim 23, wherein the modulation results in the generation of nucleic acid amplicons that are smaller compared to nucleic acid amplicons generated in a reference nucleic acid amplification using a polymerase with strand displacement activity without inorganic lithium and/or magnesium salt.

25: The method according to claim 23, wherein the modulation results in the generation of nucleic acid amplicons that are less branched compared to nucleic acid amplicons generated in a reference nucleic acid amplification using a polymerase with strand displacement activity without inorganic lithium and/or magnesium salt.

26: The method according to claim 23, wherein said inorganic lithium salt is selected from the group consisting of lithium chloride (LiCl), lithium oxide (Li₂O), lithium bromide (LiBr), lithium iodide (LiI), lithium carbonate (Li₂CO₃), lithium sulfite (Li₂SO₃), lithium sulfate (Li₂SO₄), lithium phosphate (Li₃PO₄), and lithium nitrate (LiNO₃).

27: The method according to claim 23, wherein said inorganic magnesium salt is selected from the group consisting of magnesium chloride (MgCl), magnesium oxide (MgO), magnesium bromide (MgBr₂), magnesium iodide (MgI₂), magnesium carbonate (MgCO₃), magnesium sulfite (MgSO₃), magnesium sulfate (MgSO₄), magnesium phosphate (Mg₃(PO₄)₂), and magnesium nitrate (Mg(NO₃)₂).

28: The method according to claim 23, wherein said nucleic acid is double-stranded desoxyribonucleic acid (dsDNA) and/or double-stranded ribonucleic acid (dsRNA).

29: The method according to claim 23, wherein said polymerase with strand displacement activity is selected from the group consisting of φ29 DNA polymerase, Bst DNA Polymerase, Bst DNA Polymerase large fragment, and Taq DNA polymerase mutant (SD DNA polymerase).

30: The method according to claim 23, wherein said nucleic acid amplification is whole genome amplification (WGA).

31: The method according to claim 23, wherein said inorganic lithium salt and/or inorganic magnesium salt is used in a concentration range from about 1 mM to 100 mM.

32: A method of amplifying nucleic acid comprising:
providing a nucleic acid in a reaction environment; and
amplifying said nucleic acid in the reaction environment;
wherein said reaction environment further comprises a polymerase with strand displacement activity and an inorganic lithium salt and/or an inorganic magnesium salt.

33: The method according to claim 32, wherein said inorganic lithium salt is selected from the group consisting of lithium chloride (LiCl), lithium oxide (Li₂O), lithium bromide (LiBr), lithium iodid (LiI), lithium carbonate (Li₂CO₃), lithium sulfite (Li₂SO₃), lithium sulfate (Li₂SO₄), lithium phosphate (Li₃PO₄), and lithium nitrate (LiNO₃).

34: The method according to claim 32, wherein said inorganic magnesium salt is selected from the group consisting of magnesium chloride (MgCl), magnesium oxide (MgO), magnesium bromide (MgBr₂), magnesium iodid (MgI₂), magnesium carbonate (MgCO₃), magnesium sulfite (MgSO₃), magnesium sulfate (MgSO₄), magnesium phosphate (Mg₃(PO₄)₂), and magnesium nitrate (Mg(NO₃)₂).

35: The method according to claim 32, wherein said nucleic acid is double-stranded desoxyribonucleic acid (dsDNA) and/or double-stranded ribonucleic acid (dsRNA).

36: The method according to claim 32, wherein said polymerase with strand displacement activity is selected from the group consisting of φ29 DNA polymerase, Bst DNA polymerase, Bst DNA polymerase large fragment, and Taq DNA polymerase mutant (SD DNA polymerase).

37: The method according to claim 32, wherein said nucleic acid is whole genome nucleic acid and said amplifying is whole genome amplification (WGA).

38: The method according to claim 32, wherein said reaction environment comprises the inorganic lithium salt and/or the inorganic magnesium salt in a concentration range from about 1 mM to 100 mM.

39: A method of preparing a nucleic acid sequencing library comprising:
providing an amplified nucleic acid; and
subjecting said amplified nucleic acid to a nucleic acid sequencing library;
wherein said amplified nucleic acid is prepared according to the method of claim 32.

40: A method of sequencing a nucleic acid comprising:
providing an amplified nucleic acid and
subjecting said amplified nucleic acid to a sequencing reaction;
wherein said amplified nucleic acid is prepared according to the method of claim 32.

41: A kit for amplifying nucleic acid comprising:
a first container comprising (i) a polymerase with strand displacement activity,
(ii) an inorganic lithium salt and/or an inorganic magnesium salt, and
(iii) random and/or target-specific primers, each in solution or in lyophilized and/or crystalline formulation;
optionally, a second container containing a diluent or reconstituting solution for the lyophilized fand/or crystalline formulation; and
optionally, instructions for carrying out the method according to claim 32.

42: The kit according to claim 41, wherein said inorganic lithium salt is selected from the group consisting of lithium chloride (LiCl), lithium oxide (Li₂O), lithium bromide (LiBr), lithium iodid (LiI), lithium carbonate (Li₂CO₃), lithium sulfite (Li₂SO₃), lithium sulfate (Li₂SO₄), lithium phosphate (Li₃PO₄), and lithium nitrate (LiNO₃).

43: The kit according to claim 41, wherein said inorganic magnesium salt is selected from the group consisting of magnesium chloride (MgCl), magnesium oxide (MgO), magnesium bromide (MgBr₂), magnesium iodid (MgI₂), magnesium carbonate (MgCO₃), magnesium sulfite (MgSO₃), magnesium sulfate (MgSO₄), magnesium phosphate (Mg₃(PO₄)₂), and magnesium nitrate (Mg(NO₃)₂).

44: The kit according to claim 41, wherein said polymerase with strand displacement activity is selected from the group consisting of φ29 DNA polymerase, Bst DNA polymerase, Bst DNA polymerase large fragment, and Taq DNA polymerase mutant (SD DNA polymerase).
